(19) United States
(12) Patent Application Publication
(10) Pub. No.: US 2002/0120214 A1
(43) Pub. Date: Aug. 29, 2002

(54) Spatula for biological sampling

(75) Inventor: Robert B. Cole, Alamo, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Robert B. Cole, Alamo, CA

(21) Appl. No.: 10/026,373

(22) Filed: Dec. 21, 2001

Related U.S. Application Data
(60) Provisional application No. 60/259,260, filed on Dec. 29, 2000.

(51) Int. Cl. A61B 10/00

(52) U.S. Cl. 600/570; 600/569

(57) ABSTRACT

Spatulas for biological sampling. The spatulas of the present invention include an elongated stem formed as a flat strip of uniform width, which has at one end thereof a flat scraper having a slit or an opening at about the center of the flat scrapper. At the other end, the elongated stem has either a second flat scraper or, alternatively, forms a rod-like handle.

### CROSS-REFERENCES TO RELATED APPLICATIONS

This application claims the benefit of U.S. Provisional Application No. 60/259,260 (Attorney docket No. 20064-000100US), filed Dec. 29, 2000, the disclosure of which is incorporated herein by reference in its entirety.

### FIELD OF THE INVENTION

The present invention relates to spatulas for biological sampling. More specifically, the present invention relates to spatulas for obtaining cytologic samples and, in particular, to spatulas for use with gynecological procedures (such as the Papanicolaou (“Pap”) smear test), where samples of body secretions, especially from the cervix, e.g., the ectocervix and the endocervix, are obtained and examined to detect the presence of abnormal cells (e.g., cancerous cells).

### BACKGROUND OF THE INVENTION

It has become axiomatic that the best time to treat diseases, such as cervical cancer, is when they are in their earliest phases. By necessity, the early treatment of such diseases requires that they be detected early. A variety of known techniques for detecting such diseases entail the scraping or sampling of tissue from the ectocervix or endocervical canal. Tissue so obtained can be subjected to cytologic or other examinations, perhaps the most common of which is the Pap Smear test. The accuracy of the Pap Smear is dependent upon the retrieval of a maximum number of cells from both the endocervix and ectocervix, giving the cytotechnologist the best representation of the cells present on the surface of the cervix. For the Pap Smear to be most accurate, a maximum number of cells must be both exfoliated (i.e., scraped from the tissues themselves) and retrieved from the exfoliating device for cytologic evaluation, utilizing either a slide or recently developed liquid-based cytologic technologies. Failure to obtain an adequate amount of endocervical cells results in an inadequate cytologic sampling for interpretation, rendering the Pap Smear non-diagnostic. Unfortunately, this requires that the physician repeat the Pap Smear, necessitating that the patient return to the office and, thus, duplicating the costs of the physical examination and the cytologic examination and interpretation. Repeated Pap Smears due to an inadequate amount of endocervical cells being obtained result in a significant healthcare expenditure.

The known devices for ectocervical and endocervical tissue exfoliation and collection vary widely in complexity and utility. The most simple is a cotton swab that is introduced into the uterine canal. Wet spatulas or, alternatively, plastic spatulas have also been used to sample the ectocervical and the endocervical canal. Tissue samples have been aspirated through plastic or glass pipettes. Curettage of the endocervical canal has been performed utilizing various curettes. More recently, certain brushes have been specially adapted for the exfoliation or collection of cellular material. A principal function of these brush devices is the collection of endocervical cells which are particularly diagnostic in the evaluation of the Pap Smear. There have been no special adaptations, however, for the facilitation of retrieval of collected cellular material. In fact, to date, no simple and practical method or device has been developed that ensures maximum recovery of endocervical cells collected from these brush devices.

As such, what is needed in the art is a method and device that simultaneously possess the ability to exfoliate cervical and endocervical cellular material and the ability to maximize retrieval of the collected cellular material from these brush devices for cytological evaluation. The present invention advantageously fulfills these and other needs.

### SUMMARY OF THE INVENTION

The present invention provides methods and devices for biological sampling and retrieval. More particularly, the present invention provides methods and inexpensive, simple instruments, i.e., spatulas, for obtaining endocervical cells in much greater quantities and better quality than, for example, the previously used methods and devices. The spatulas of the present invention can be employed in a way that significantly increases cytodiagnostic accuracy by an increased quantitative and qualitative improvement of the cell yield, which, in turn, increases the sensitivity of the cellular sample taken. Use of the spatulas of the present invention allows the physician to make an early diagnosis of significant cytopathologic abnormalities. Additionally, by improving the likelihood of retrieval of endocervical cells, the necessity for repeating cytologic evaluations is reduced, thereby significantly reducing the number of repeat “Pap Smears” that must be performed as a result of a failure to retrieve adequate numbers of endocervical cells, and thereby also saving significant healthcare costs.

The present invention provides a spatula having significant advantages over prior devices. Most notably, the spatula of the present invention has a good ability to exfoliate tissue and is designed to work in conjunction with brush cytologic collection devices to maximize retrieval of collected cellular material. This combination of exfoliating and collecting abilities is achieved even though the spatula is easy to use and is non-invasive, is well tolerated by patients, and is safe and cost-effective. Such advantages make the spatula of the present invention useful in a wide variety of clinical applications. For instance, new liquid-based cytologic systems for “Pap Smear” collection require maximum transfer of cells collected from the cervix to the liquid medium. The spatula of the present invention allows for the rapid and effective transfer of cellular material collected from brush systems to such liquid-based cytologic systems. It has been found that the new technologies for Pap Smear collection and evaluation dramatically improve cytologic evaluation of the cells obtained. Improved interpretation of the “Pap Smear” allows for earlier diagnosis of pathologic conditions and allows for clearer interpretation of the etiology of cellular abnormalities revealed in the smear. Greater reliability of endocervical collection also decreases the need for repetitive tests to obtain an accurate diagnosis. As such, by increasing the reliability of endocervical collection, the spatula of the present invention makes these new technologies for Pap Smear collection and evaluation even that much more accurate and reliable.

There are multiple pathologic states present in the female genital tract which require early diagnosis and treatment. Both human papilloma virus and cervical dysplasia require the earliest possible diagnosis and treatment. Unfortunately, there are a wide range of equivocal interpretations in “Pap Smears.” These interpretations fall into the diagnosis of “atypical Pap Smears.” This represents a large gray zone in which the clinician is required to repeat the Pap Smear at 3 to 6 month intervals. In many cases, there is an underlying pathologic condition that is going untreated while these repeat studies are being performed. Larger cell recoveries specifically utilizing the new liquid-based cytologic Pap Smear technologies have been shown to reduce the number of “atypical Pap Smears,” thereby allowing the practitioner to move forward with a diagnosis of a real pathologic state or to reassure the patient that there is no disease process to be concerned about. Clearly, this is beneficial both to the patient psychologically and to our health care system in that it reduces the number of unnecessary “Pap Smears” and expedites the time to diagnosis and treatment. The Pap Smear continues to be one of the most widely performed cytologic procedures in the world and is the cornerstone to reducing the frequency of invasive cervical cancer which is a devastating disease and continues to kill thousands of women annually. Improving diagnosis through the use of the spatula of the present invention represents a significant advance in women's health care.

In essence, the spatula of the present invention represents a modification of the standard cervical scraper that allows the scraper to work directly in conjunction with an endocervical cytologic brush. The spatula design involves the placement of a slit or an opening within the working end of the spatula that exfoliates cellular debris from the ectocervix. Moreover, the slit or opening in the spatula allows for the manipulation of the cervical mucus that has been collected from the cytologic endocervical brush and allows for easy removal of mucus and cellular material that has been collected from the endocervical canal. The cytobrush can be drawn through the slit which has been placed in one of several locations on the spatula end, thereby allowing the practitioner to remove all of the material that is adherent to the bristles of the brush. The location and configuration of the slit(s) or opening(s) can vary. However, the essence of the spatula of the is that it contains a slit or an opening that allows for the cytobrush to be divested of its collected cellular material.

In one embodiment, a spatula of the present invention includes an elongated stem formed as a flat strip of substantially uniform width, which has at one end thereof a flat scraper portion. The flat scraper preferably includes a slit and/or an opening therein. At the other end, the elongated stem has either a second flat scraper portion or, alternatively, forms a rod-like handle. It is noted that, if present, the second flat scraper portion can optionally have a second opening or slit, e.g., at about the center of the second flat scraper portion. In a presently preferred embodiment, a star-shaped or key-shaped opening, either alone or in combination with a slit, is present in roughly the center of a flat scraper portion to facilitate the removal of the cellular material from the brush. In a presently preferred embodiment, the spatula is fabricated from any semi-rigid material, such as a plastic material.

According to an aspect of the present invention, a spatula for biological sampling is provided that typically includes an elongated stem having a flat scraper at one end of the elongated stem, wherein the flat scraper has a slit at about the center of the flat scraper.

According to another aspect of the present invention, a spatula for biological sampling is provided that typically includes an elongated stem having a scraper portion at one end of the elongated stem, wherein the flat scraper has an opening defined therein.

According to yet another aspect of the present invention, a spatula for biological sampling in combination with a cytobrush is provided. The cytobrush typically has a plurality of bristles at a first end of a handle, the bristles for collecting cellular material. The spatula typically includes an elongated stem having a flat scraper portion at one end, the flat scraper portion including an opening defined therein having a first portion of sufficient size to receive the first end of the cytobrush without substantially disturbing the bristles and a second portion having a smaller size such that when the cytobrush is inserted into the first portion and removed through the second portion, a substantial portion of the cellular material on the bristles is removed onto the flat scraper portion.

According to a further aspect of the present invention, a method is provided for collecting cellular material on a spatula having a flat scraper portion at one end of an elongated stem. The flat scraper portion of the spatula includes an opening defined therein having a first portion of sufficient dimension to receive the first end of a cytobrush without substantially disturbing the bristles of the cytobrush, and a second portion having a smaller dimension than the first portion. The method typically includes collecting cellular material on the bristles of the cytobrush, inserting the first end of the cytobrush into the first portion of the opening, and removing the first end of the cytobrush through the second portion of the opening whereby a substantial portion of the cellular material on the bristles is removed onto the flat scraper portion.

According to yet a further aspect of the present invention, a method is provided for collecting cellular material on a spatula having a flat scraper portion at one end of an elongated stem. The flat scraper portion of the spatula includes an opening defined therein and extending to the perimeter of the scraper portion, with the opening being of sufficient width to receive the handle of a cytobrush. The method typically includes collecting cellular material on the bristles of the cytobrush, wherein the handle of the cytobrush has a cross-sectional area smaller than the cross-sectional area of the bristles, inserting the handle of the cytobrush into the opening, and removing the bristles of the cytobrush through the opening whereby a substantial portion of the cellular material on the bristles is removed onto the flat scraper portion.

Other features and advantages of the present invention will become apparent from the following description, taken in conjunction with the accompanying drawings.

### BRIEF DESCRIPTION OF THE DRAWINGS

FIG. 1 is a plan view of a spatula having a flat scraper 11 at one end and a flat scraper 12 at the other end, wherein the flat scraper 11 has a slit 8 at about the center of the scraper.

FIG. 2 is a plan view of a spatula having a flat scraper 11 at one end and a flat scraper 12 at the other end, wherein the flat scraper 12 has a slit 9 at about the center of the scraper.

FIG. 3 is a plan view of a spatula having a flat scraper 11 at one end and a flat scraper 12 at the other end, wherein the flat scraper 11 has a slit 8 at about the center of the scraper and both the flat scrapers 11 and 12 include perforations around the perimeters.

FIG. 4 is a plan view of a spatula having a flat scraper 11 at one end and a flat scraper 12 at the other end, wherein the flat scraper 12 has a slit 8 at about the center of the scraper and both the flat scrapers 11 and 12 include perforations around the perimeters.

FIG. 5 is a side view of the spatulas of the present invention.

FIG. 6 is a plan view of a spatula having a flat scraper 11 at one end and a flat scraper 12 at the other end, wherein the flat scraper 11 has a slit 8 located on the larger lobe of the lateral aspect of the flat scraper 11.

FIG. 7 is a plan view of a spatula having a flat scraper 11 at one end and a flat scraper 12 at the other end, wherein the flat scraper 11 has a slit 8 located on the smaller lobe of the lateral aspect of the flat scraper 11.

FIG. 8 is a plan view of a spatula having a flat scraper 11 at one end and a flat scraper 12 at the other end, wherein the flat scraper 12 has a slit 9 located along the side of the flat scraper 12.

FIG. 9 is a plan view of a spatula having a flat scraper 11 at one end and a flat scraper 12 at the other end, wherein the flat scraper 11 has an opening 24 in roughly the center of the flat scraper 11.

FIG. 10 is a plan view of a spatula having a flat scraper 11 at one end and a flat scraper 12 at the other end, wherein the flat scraper 11 has an opening 25 in roughly the center of the flat scraper 12.

### DETAILED DESCRIPTION OF THE INVENTION AND PREFERRED EMBODIMENTS

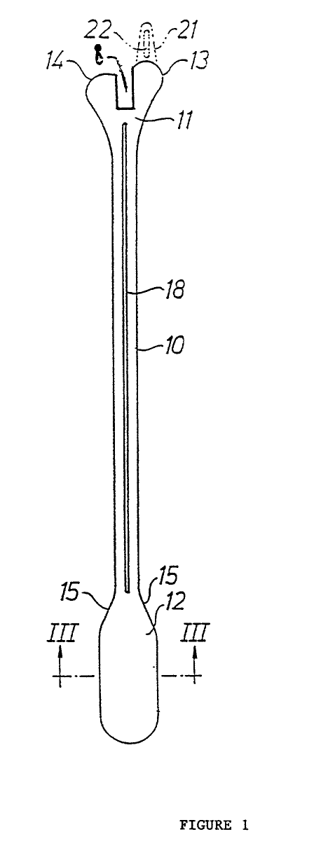

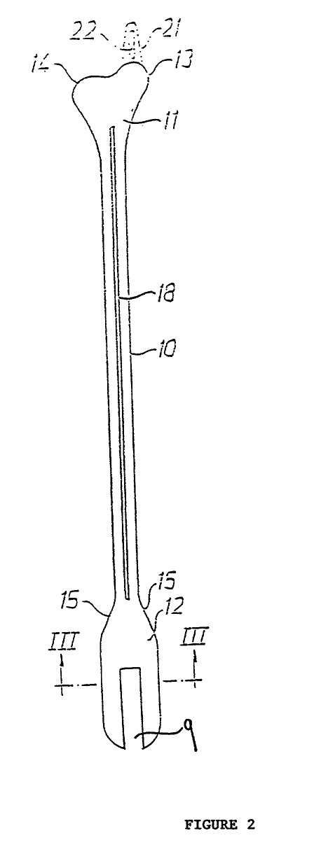

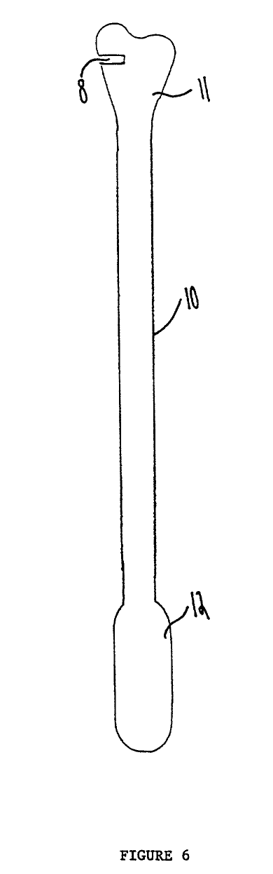

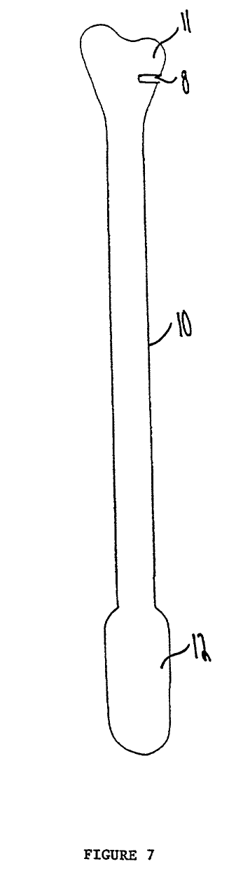

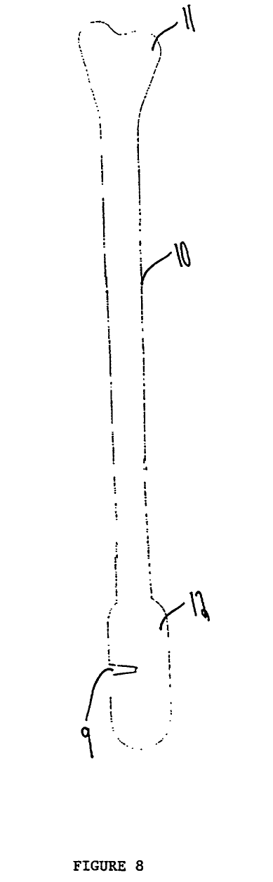

The present invention provides spatulas for biological sampling. More particularly, the present invention provides methods and inexpensive, simple instruments, i.e., spatulas, for obtaining endocervical cells in much greater quantities and better quality than previously used methods and devices. The spatulas of the present invention, which are illustrated in FIGS. 1-10, each comprise an elongated stem 10, preferably formed as a flat strip of substantially uniform width, which has at one end thereof a flat scraper portion 11 having a slit 8, or an opening 24 at about the center of the flat scrapper 11 (or both a slit and an opening). At the other end, the elongated stem has either a second flat scraper portion 12 or, alternatively, forms a rod-like handle with no additional scraper portion. It is noted that, if present, the second flat scraper 12 can optionally have a second slit or a second opening or both a second slit and a second opening.

It will be readily apparent to those of skill in the art that the slit(s) can run throughout the length of the flat scraper(s) or, alternatively, throughout a portion of the length of the flat scraper(s). It is also readily apparent to those of skill that a slit can be in various locations within the scraper, e.g., central or lateral or both. For example, in one embodiment, the slit runs substantially throughout the length of the flat scraper portion aligned with the axis of shaft 10. In addition, it will be readily apparent to those of skill in the art that the slit(s) can be of uniform width or, alternatively, of varying width. Typically, each slit is of a width sufficient to permit a cytobrush to be drawn through it such as to remove a substantial portion of the cellular material collected on the brush. In a preferred embodiment, the slit is of varying width, with the widest end of the slit being from about 1 mm to about 8 mm wide and, more preferably, from about 2 mm to about 4 mm wide. Additionally, an opening, in, for example, a star shape, a key shape or some other shape, can be present which would work in conjunction with, or lieu of, the slit. Typically, the opening is located in the center of the flat scraper and is of a size sufficient to permit a cytobrush to be drawn through it.

In one embodiment of the present invention, as shown in the Figures, the elongated stem 10 has at one end thereof a first flat scraper portion 11 and at the other end thereof a second flat scraper portion 12. The two scraper portions (“scrapers”) are preferably wider than the elongated stem, have substantially the same thickness as the stem—on the order of 1 to 2 mm or more—and are coplanar with the elongated stem. First scraper 11 is substantially heart-shaped and forms two lobes 13 and 14 projecting at an oblique angle to the longitudinal axis of the stem. The longitudinal axes of the lobes are preferably substantially perpendicular to each other and converge towards, and merge on, the longitudinal axis of the stem 10. In one embodiment, as shown, lobe 13 is longer than the lobe 14, and the two lobes have circularly convex ends. The second scraper 12 is substantially rectangular with the side edges extending in parallel with the stem and having a circularly convex end. Oblique shoulders 15 join second scraper 12 to the stem 10.

Although first scraper portion 11 is shown as being lobed and (optional) second scraper portion 12 is shown as being rectangular with rounded ends, and shoulders to shaft 10, it should be appreciated that each of first scraper portion 11 and (optional) second scraper portion 12 may be lobed, rectangular or defined by some other useful shape.

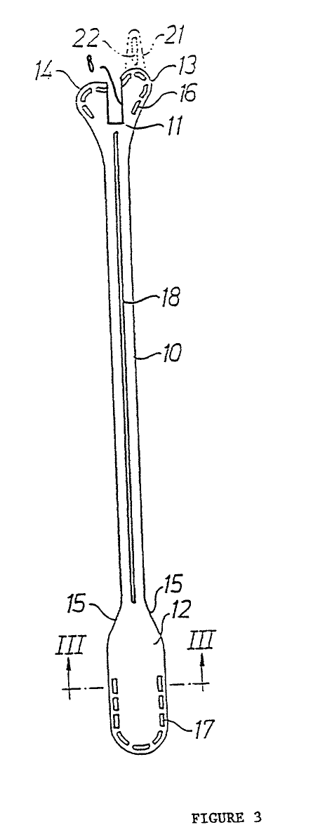

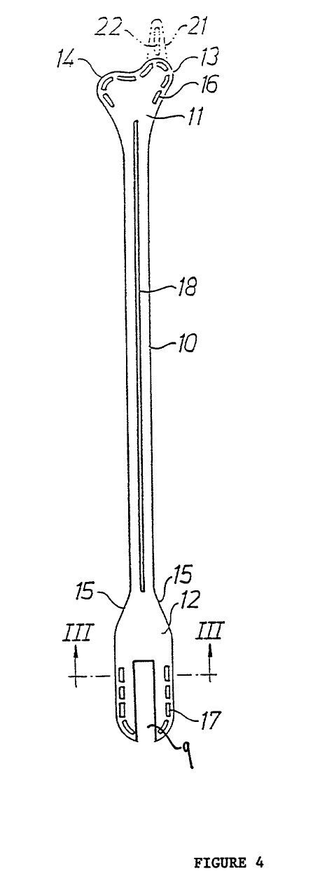

In another embodiment of the present invention as shown in FIGS. 3 and 4, perforations are provided along the edges of one or both scraper(s). When perforations are provided in the flat scraper, it has been found that the scraped-off sample is more readily retained on the spatula by capillary action in the perforations, conditioned by the surface tension of the sample, without preventing the cellular sample from being released when the sample is being smeared onto a slide or washed off in a transport solution. For instance, in one embodiment as shown in FIGS. 3 and 4, perforations 16 are provided in the flat scraper portion 11 and follow the contour of the edge of the scraper along the edge of the lobes 13 and 14. The perforations include a series of mutually separated slots. In a similar manner, the (optional) second flat scraper 12 includes perforations 17 which also include a series of mutually separated slots extending along the edge of the flat scraper 12, but only in a portion thereof which is close to the free end of the flat scraper as shown. The slots each preferably have a width of less than about 2 mm and a length of less than about 6 mm and more preferably a width of about 1 mm and a length of about 4 mm.

The perforations are preferably formed by substantially rectangular slots, but it will be readily apparent to those of skill in the art that the perforations can also be formed by circular, triangular, square or polygonal apertures. The perforations can also be arranged in a manner other than along the edge of the flat scraper.

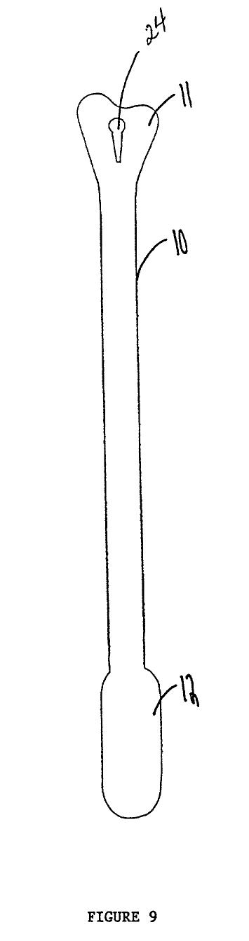

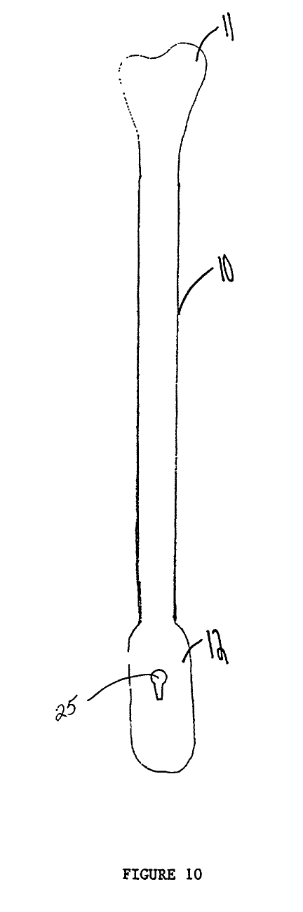

Another embodiment of the present invention is shown in FIGS. 9 and 10. As shown in FIG. 9, the spatula includes an elongated stem 10 formed as a flat strip of substantially uniform width, which has at one end thereof a flat scraper portion 11 having an opening 24 at about the center of the flat scrapper (or both an opening and a slit). At the other end, the elongated stem has either a second flat scraper 12 or, alternatively, forms a rod-like handle. As shown in FIG. 10, the spatula includes a flat scraper portion 12 having an opening 25 at about the center of the scraper. At the other end the elongated stem 10 either forms another flat scraper 11 or forms a rod like handle. It should be appreciated that where the spatula includes flat scraper portions at each end, both scraper portions may include an opening and/or a slit. Typically, an opening, (e.g., opening 24 or 25) is located in the center of the flat scraper and is of a size sufficient to permit a cytobrush to be drawn through it (typically, from about 1 mm to about 8 mm in diameter and, more preferably, from about 2 mm to about 4 mm in diameter) such as to remove a substantial portion of the cellular material collected thereon. The opening can be of any shape and, preferably, is a star-shaped or key-shaped opening. This allows the user to insert the brush into the larger portion of the opening without disrupting much cellular material collected thereon, move the (narrower) handle of the brush into the narrower portion of the opening and pull the brush out, thereby removing a substantial portion of the cellular material collected thereon.

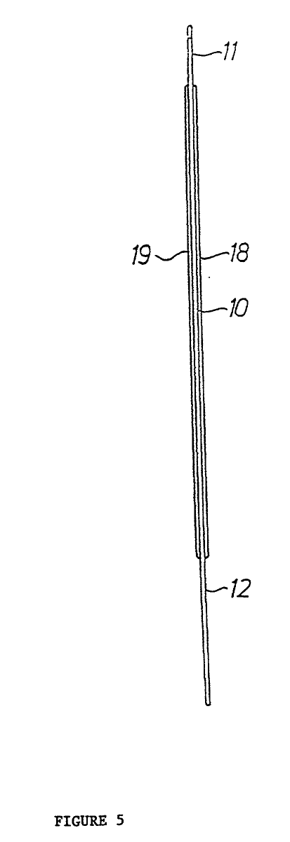

In yet another embodiment of the present invention, with reference to FIG. 5, the elongated stem 10 is stiffened by means of at least one stiffening rib 18 or 19. In a preferred embodiment, each side of the strip forming the elongated stem is stiffened by means of a stiffening rib 18 and 19, respectively, extending along the center line of the strip over a substantial length of the stem. It will be readily apparent to those of skill in the art that the stiffening ribs can be replaced by edge flanges provided on the strip so that it is H-shaped in cross-section.

The spatulas of the present invention are typically made in a single piece by injection molding of a suitable plastic material (i.e., the spatula is one-piece molded). In a presently preferred embodiment, the plastic material is of such a kind that the spatula is flexible. Suitable plastic materials include, but are not limited to, polypropylene, polyethylene, polyamide (i.e., nylon) and polystyrene. In a presently preferred embodiment, polypropylene is the plastic material used to make the spatula of the present invention.

It is noted that the flexibility of the spatula or at least the flat scraper 11 and/or 12 must be adjusted and controlled so that the flat scraper can adapt itself to the anatomic conditions for access to the sampling region and the cellular sample can be smeared onto the slide, which requires a certain flexibility of the flat scraper. However, the flexibility of the spatula in its entirety must not be so great that the elongated stem is too weak and will be twisted when the spatula is rotated during sampling.

In another embodiment of the present invention, at least one side of a flat scraper 11 and/or 12 has a matted or frosted surface. When the flat scraper has a matted or frosted surface, it has been found that the adhesion of the cellular sample to the spatula is improved. In a presently preferred embodiment, both sides of a flat scraper have a matted or frosted surface. It has further been found that the adhesion of the cellular sample to the spatula can be improved by increasing the roughness of the matted or frosted surface.

The spatulas of the present invention can be used in a wide variety of ways, and the following embodiments serve to illustrate several ways in which the spatulas of the present invention can be used. It is noted that such embodiments are illustrative and not restrictive. In one embodiment, the spatula is used to collect the sample from the ectocervix, the flat scraper 11 being used for the sampling. At the sampling, the spatula is introduced into the vagina that is kept widened by means of a speculum, and the lobe 13 is engaged with the external os. Then, the spatula is rotated at least one turn so that the two lobes 13 and 14 of the flat scraper 11 scrape off cells and secretions from the external os and the ectocervix. The collected cells together with secretion are retained on the spatula, but can easily be smeared onto a slide or are washed off in a transport solution.

In another embodiment, the spatula is used to collect the sample from the ectocervix using the flat scraper 11 as described above. However, once the collected cells have been retained on the spatula, a cytobrush (such as those disclosed and claimed in U.S. Pat. Nos. 4,759,376, 5,713,369 and 5,623,941, the teachings of which are incorporated herein by reference) is used to remove or collect the cells from the spatula. This can be accomplished by brushing both sides of the spatula with the cytobrush—either once or a multiple of times. Once the collected cells are on the cytobrush, the cytobrush is drawn through a slit, e.g., slit 8 or 9, or an opening, e.g., opening 24 or 25, and the collected cells are concentrated in one region of the spatula. Thereafter, the collected cells can easily be smeared onto a slide or be washed off in a transport solution.

In yet another embodiment, both the spatula of the present invention and a cytobrush are used to collect the sample from the ectocervix. The spatula is used as described above. The cytobrush is used in an analogous manner. More particularly, at the sampling, the cytobrush is introduced into the vagina, which is kept widened by means of a speculum, with the narrow end of the conically shaped tip entering the endocervical canal until a resistance is felt. The brush is then turned by the operator one-half to about two complete turns and then slowly extracted with the mucous and cellular samples within the bristles. Once the cells have been collected, the cytobrush may also be used to remove the cells from the spatula. Again, this can be accomplished by brushing both sides of the spatula with the cytobrush—either once or a multiple of times. Once the collected cells are on the cytobrush, the cytobrush is drawn through a slit or opening, and the collected cells are concentrated in one region of the spatula. Thereafter, the collected cells can easily be smeared onto a slide or be washed off in a transport solution.

In addition to the foregoing, it has been discovered that by forming an axially, i.e., along the direction of the axis of stem 10, projecting tip on the lobe 13 as indicated by a dot-and-dash line 21 in FIGS. 1-4, the sampling from the ectocervix can be combined with sampling from the endocervical canal by the tip 21 being inserted therein. Also the tip 21 can include a perforation and is shown herein with a longitudinal slot 22.

For sampling from the upper part of the vagina (fornix) the flat scraper 12 is preferably used. The collected cellular sample is treated as described above.

It is important to note that the sample material collected using the spatulas and methods of the present invention can be used for both cytological and histological diagnosis. Histology is performed from the fragments of tissue yielded by the spatula or spatula/cytobrush combination. Tissue fragment histology increases the diagnostic accuracy of the collecting procedure. Cytology, in contrast, allows the physician to make a clinical decision regarding the condition of the patient. The spatulas (or spatula/cytobrush combination) of the present invention facilitate both of these because it retrieves cytological samples that are more accurate than the samples obtained with the standard sampling products on the market today.

With regard to morphologic evaluation, the spatulas (or spatula/cytobrush combination) of the present invention provide tissue samples (e.g., endocervical tissue samples) very well suited for the recognition of abnormal cells associated with a variety of conditions or circumstances, including, but not limited to, the following: those associated with or consistent with benign atypia (non-hyperplastic), such as acute inflammation, chronic inflammation, granulomatous inflammation, squamous metaplasia and irradiation effects; those associated with or consistent with cervical dysplasia, either mild, moderate or severe; and those associated with or consistent with malignancy, such as squamous cell carcinoma of the cervix, adenocarcinoma of the cervix, mixed adenosquamous carcinoma of the cervix, carcinoma in situ of the cervix, vaginal squamous cell carcinoma; as well as the recognition of other abnormal cells not specifically set forth herein.

Although the spatulas of the present invention have been described herein in connection with cytological sampling, it will be readily apparent to those of skill in the art that such spatulas can be used for cell sampling from other mucous areas as well as erosions or ulcers of the skin. In addition, the spatulas of the present invention can also be used for microbiological sampling. Further, it is understood that such spatulas may be provided in combination with a cytobrush, either separately or integrally connected, e.g., using a cord, wire or other flexible connector.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A spatula for biological sampling, said spatula comprising an elongated stem having a flat scraper at one end of said elongated stem, wherein said flat scraper has a slit at about the center of said flat scraper.

2. The spatula in accordance with claim 1, wherein said slit extends through the length of said flat scraper.

3. The spatula in accordance with claim 1, wherein said slit is of substantially uniform width.

4. The spatula in accordance with claim 1, wherein said slit has a varying width.

5. The spatula in accordance with claim 4, wherein the widest portion of said slit has a width of about 2 millimeters to about 4 millimeters.

6. The spatula in accordance with claim 4, wherein the widest portion of said slit has a width of less than about 8 millimeters.

7. The spatula in accordance with claim 1, further comprising one or more perforations within said flat scraper and along the edge of said flat scraper.

8. The spatula in accordance with claim 1, further comprising a plurality of elongated slots along the edge of said flat scraper and following the contour of the edge of said flat scraper.

9. The spatula in accordance with claim 8, wherein said slots each have a width of less than about 2 mm.

10. The spatula in accordance with claim 8, wherein said slots each have a length less than about 6 mm.

11. The scraper in accordance with claim 1, further comprising a plurality of apertures along the edge of said flat scraper, each of said apertures having one of a circular, triangular, rectangular, square and polygonal shape.

12. The spatula in accordance with claim 11, wherein said apertures each have a dimension of less than about 2 mm.

13. The spatula in accordance with claim 11, wherein said apertures each have a dimension of less than about 6 mm.

14. The spatula in accordance with claim 1, wherein said flat scraper is substantially heart-shaped having two lobes projecting from said elongated stem, each lobe at an oblique angle relative to the longitudinal axis of said elongated stem, and having a circularly convex end.

15. The spatula in accordance with claim 14, wherein one lobe projects farther than the other lobe.

16. The spatula in accordance with claim 15, wherein the longer lobe includes a tip formed thereon for sampling from the endocervical canal, wherein said tip extends from the end of the longer lobe substantially along the direction of the axis of said stem.

17. The spatula in accordance with claim 14, wherein the axes of said lobes converge toward the axis of said elongated stem.

18. The spatula in accordance with claim 17, wherein the axes of said lobes are substantially perpendicular to each other.

19. The spatula in accordance with claim 1, wherein said flat scraper is made of a plastic material.

20. The spatula in accordance with claim 19, wherein said plastic material is a polymeric material selected from the group consisting of polypropylene, polyethylene polyamide and polystyrene.

21. The spatula in accordance with claim 1, further comprising a second flat scraper is provided at the other end of said elongated stem, wherein the second flat scraper has a substantially rectangular shape and a circularly convex end.

22. The spatula in accordance with claim 21, wherein said elongated stem has substantially the same width as said flat scrapers.

23. The spatula in accordance with claim 1, wherein said elongated stem has a substantially uniform width.

24. The spatula in accordance with claim 1, wherein said elongated stem has substantially the same width as said flat scraper.

25. The spatula in accordance with claim 1, further including at least one stiffening rib on one side of said elongated stem, and extending along said elongated stem.

26. The spatula in accordance with claim 1, further including a stiffening rib on each side of said elongated stem, each rib extending along said elongated stem.

27. The spatula in accordance with claim 1, wherein at least one side of said flat scraper has a matted surface.

28. The spatula in accordance with claim 1, wherein both sides of said flat scraper have a matted surface.

29. The spatula in accordance with claim 1, wherein said spatula is made of a flexible material.

30. The spatula in accordance with claim 1, wherein said flat scraper has a thickness of about 1 mm.

31. The spatula in accordance with claim 1, wherein said flat scraper has a thickness of less than about 2 mm.

32. A spatula for biological sampling, said spatula comprising an elongated stem having a scraper portion at one end of said elongated stem, wherein said flat scraper has an opening defined therein.

33. The spatula of claim 32, wherein said opening is proximal the center of said scraper.

34. The spatula of claim 33, wherein said opening is completely contained within said flat scraper portion.

35. The scraper of claim 34, wherein said opening includes a first portion having a first dimension sufficient to receive the bristles of a cytobrush without substantially disturbing the bristles, and a second portion having a second dimension smaller than the first dimension such that when the cytobrush is removed through the second portion, a substantial portion of any cellular material on the bristles is removed onto the flat scraper portion.

36. The spatula of claim 32, wherein said opening extends inwards from an edge of said scraper portion.

37. The scraper of claim 36, wherein the opening is of sufficient dimension to receive the handle of a cytobrush from the edge of the flat scraper portion and such that a substantial portion of cellular material on the bristles is removed onto the flat scraper portion when the bristles of the cytobrush are removed through the opening.

38. The scraper of claim 32, wherein said opening is one of a key-shaped opening and a star-shaped opening.

39. A spatula for biologic sampling in combination with a cytobrush having a plurality of bristles at a first end of a handle, said bristles for collecting cellular material, the spatula comprising an elongated stem having a flat scraper portion at one end, said flat scraper portion including an opening defined therein having a first portion of sufficient size to receive the first end of the cytobrush without substantially disturbing the bristles and a second portion having a smaller size such that when the cytobrush is inserted into the first portion and removed through the second portion, a substantial portion of the cellular material on the bristles is removed onto the flat scraper portion.

40. A method of collecting cellular material on a spatula having a flat scraper portion at one end of an elongated stem, said flat scraper portion including an opening defined therein having a first portion of sufficient dimension to receive the first end of a cytobrush without substantially disturbing the bristles of said cytobrush and a second portion having a smaller dimension than the first portion, the method comprising:
collecting cellular material on the bristles of a cytobrush;
inserting the first end of the cytobrush into the first portion of said opening; and
removing the first end of the cytobrush through the second portion of said opening whereby a substantial portion of the cellular material on the bristles is removed onto the flat scraper portion.

41. A method of collecting cellular material on a spatula having a flat scraper portion at one end of an elongated stem, said flat scraper portion including an opening defined therein and extending to the perimeter of the scraper portion, said opening being of sufficient width to receive the handle of a cytobrush, the method comprising:
collecting cellular material on the bristles of a cytobrush, wherein the handle of said cytobrush has a cross-sectional area smaller than the cross-sectional area of the bristles;
inserting the handle of the cytobrush into said opening; and
removing the bristles of the cytobrush through said opening whereby a substantial portion of the cellular material on the bristles is removed onto the flat scraper portion.
